(19) United States
(12) Patent Application Publication
(10) Pub. No.: US 2006/0100243 A1
(43) Pub. Date: May 11, 2006

(54) Methylphenidate analogs and methods of use thereof

(76) Inventors: Mark Froimowitz, Newton, MA (US); Charles J. Kelley, Providence, RI (US)

Correspondence Address: REED INTELLECTUAL PROPERTY LAW GROUP, 1400 PAGE MILL ROAD, PALO ALTO, CA, 94304-1124, US

(21) Appl. No.: 11/256,063

(22) Filed: Oct. 21, 2005

Related U.S. Application Data
(60) Provisional application No. 60/621,342, filed on Oct. 22, 2004.

(51) Int. Cl. C07D 211/08; A61K 31/445

(52) U.S. Cl. 514/317; 514/326; 546/192

(57) ABSTRACT

Provided are analogs of methylphenidate (“MPH”) that are useful for the treatment of drug addiction, attention deficit disorder, attention deficit hyperactivity disorder, and depression. The MPH analogs are extended duration compounds that bind to the dopamine transporter and the reuptake of dopamine in the afflicted individual's brain. Because of the extended duration of the MPH analogs, administration of the compounds is only required on a once or twice daily schedule.

### ACKNOWLEDGEMENT OF GOVERNMENT INTEREST

This invention was made with United States government support under Grant No. DA015795 awarded by the National Instituted for Drug Abuse; accordingly, the United States government has certain rights in this invention.

### TECHNICAL FIELD

This application relates generally to the field of treatments for drug addiction. More specifically, this application relates to the synthesis of methylphenidate (“MPH”) analogs that have utility as treatments for persons afflicted with addiction to drugs, in particular, dopamine reuptake inhibitors, such as cocaine. The MPH analogs of the present invention, which are also useful for the treatment of attention deficit disorder, attention deficit hyperactivity disorder, and depression, have enhanced stability over traditional MPH and thus only require once-daily administration.

### BACKGROUND OF THE INVENTION

Addiction is characterized by the compulsive use of a drug despite adverse consequences. A key problem in drug addiction is the prevention of relapse in abstinent addicts. It is well-known in the field of drug addiction that every addicting drug increases dopamine, a key neurotransmitter of the central nervous system (“CNS”). Dopamine, serotonin, and norepinephrine are three neurotransmitters in the CNS. The main classes of abused drugs are stimulants, such as amphetamines, methylphenidate, and cocaine; opiates, such as morphine, opium, and heroin; and legal drugs, such as alcohol and nicotine. Although each of these drugs influences different neurotransmitters in the brain, many drug-induced primary responses lead to increases of dopamine as secondary effects. For example, opiates first bind to an opiate receptor, which increases the activity of the mesolimbic dopamine neurons in the midbrain, which in turn increases the levels of dopamine at this site. Stimulants such as cocaine directly affect the CNS by blocking the dopamine transporter so that it is unable to remove dopamine from the synapse of dopamine neurons. As a result of increased levels of synaptic dopamine, those neurons fire longer than they would otherwise, causing a prolonged feeling of pleasure. Dopamine affects brain processes that control movement, emotional response, and the ability to experience pleasure and pain.

Within the stimulants, MPH differs most notably from cocaine in that when it is taken orally in prescribed doses, it is not addictive and does not produce the “high” characteristic of cocaine. The difference between the activities of these two dopamine reuptake inhibitors lies in the time of action of the two drugs. Specifically, while cocaine's effects on dopamine levels occur within seconds, the response from MPH, when orally administered, take much longer. Maximum drug concentration after oral administration of MPH occurs after about two hours, at which time the MPH has been absorbed from the gastrointestinal tract and has passed into the systemic circulation including the brain.

Presently, there are no known medications available to treat cocaine addiction despite extensive efforts to find such medications. Because of the similar pharmacological profiles of cocaine and MPH, the present inventors have explored synthetic analogs of MPH as potential medications for the treatment of cocaine addiction. Because the MPH analogs have slow-onsets and long-durations of action at the dopamine transporter, the MPH analogs of the present invention would be expected to have little abuse potential and could be used as a maintenance therapy for the treatment of cocaine abuse as well as for other abused drugs. Further, the MPH analogs of the present invention may also be used to treat attention deficit disorder, attention deficit hyperactivity disorder, and depression. To the best of the inventor's knowledge, the MPH analogs disclosed herein have never before been disclosed in the art.

### SUMMARY OF THE INVENTION

The present invention addresses the aforementioned needs in the art by providing a treatment for drug addiction, attention deficit disorder, attention deficit hyperactivity disorder, and depression using MPH analogs that bind to the dopamine transporter and have an extended duration of activity.

In a first embodiment of the invention, there is provided a compound having the structure of formula (I)

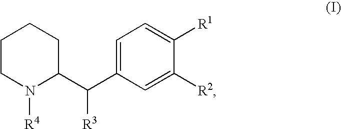

wherein:

R¹ and R² are independently selected from hydrogen, halogen, C₃-C₁₈ alkyl, alkoxy, substituted alkyl, aryl, and aralkyl, with the proviso that at least one of R¹ and R² is other than hydrogen;

R³ is selected from C₄-C₁₈ alkyl, substituted alkyl, heteroalkyl, substituted heteroalkyl, aryl, aralkyl, substituted aralkyl, heteroaralkyl, and substituted heteroaralkyl; and

R⁴ is hydrogen, alkyl, or aralkyl.

In a second embodiment of the present invention, there is a pharmaceutical composition for treating an individual suffering from drug addiction, attention deficit disorder, attention deficit hyperactivity disorder, or depression, the composition comprising a therapeutically effective amount of the compound of formula (I) and a pharmaceutically acceptable carrier:

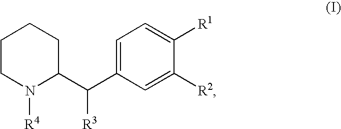

wherein:

R¹ and R² are independently selected from hydrogen, halogen, alkyl, alkoxy, substituted alkyl, aryl, and aralkyl, with the proviso that at least one of R¹ and R² is other than hydrogen;

R³ is selected from C₁-C₁₈ alkyl, substituted alkyl, heteroalkyl, substituted heteroalkyl, aryl, aralkyl, substituted aralkyl, heteroaralkyl, and substituted heteroaralkyl; an

R⁴ is hydrogen, alkyl, or aralkyl.

In a third embodiment of the present invention, there is provided a method for treating an individual suffering from drug addiction, attention deficit disorder, attention deficit hyperactivity disorder, or depression, comprising administering to the individual a therapeutically effective amount of a compound of formula (I)

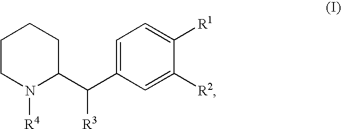

wherein:

R¹ and R² are independently selected from hydrogen, halogen, alkyl, alkoxy, substituted alkyl, aryl, and aralkyl, with the proviso that at least one of R¹ and R² is other than hydrogen;

R³ is selected from C₁-C₁₈ alkyl, substituted alkyl, heteroalkyl, substituted heteroalkyl, aryl, aralkyl, substituted aralkyl, heteroaralkyl, and substituted heteroaralkyl; and

R⁴ is hydrogen, alkyl, or aralkyl.

Within the second and third embodiment of the invention, the pharmaceutical composition and the method may be used to treat an individual addicted to a dopamine reuptake blocker, such as cocaine or methylphenidate, or to a stimulant, such as amphetamine. To treat the drug addiction, the compound may be administered orally once or twice daily.

In a fourth embodiment of the present invention, there is provided a method of synthesizing a compound for the treatment of drug addiction, attention deficit disorder, attention deficit hyperactivity disorder, or depression comprising the steps of (a) converting 1-chloro-4-bromobenzene into a Grignard reagent with magnesium and tetrahydrofuran; (b) reacting the Grignard reagent with pyridine-2-carboxaldehyde to produce an alcohol; (c) oxidizing the alcohol with pyridinium chlorochromate in methylene chloride to produce a ketone; (d) reacting the ketone with a Grignard reagent to produce an alcohol; (e) dehydrating and refluxing the alcohol with hydrogen chloride to produce an olefin; (f) hydrogenating the olefin and pyridine to produce the compound, wherein the Grignard reagent of step (d) contains functional R groups for inclusion in the compound prepared in step (f). Preferably, the compound prepared in step (f) is the compound of formula (I).

Additional embodiments, advantages and features of the invention will be set forth, in part, in the description that follows, and, in part, will become apparent to those skilled in the art upon examination of the following, or may be learned by practice of the invention.

### BRIEF DESCRIPTION OF THE DRAWINGS

FIG. 1 is a schematic that shows the synthesis of three MPH alkyl analogs of the present invention.

FIG. 2 is a graph showing the effect on mice of (1) 5 mg/kg; (2) 10 mg/kg; (3) 20 mg/kg; and 40 mg/kg of cocaine compared with saline on ambulation counts per 10 minutes over an eight-hour session.

FIG. 3 is a graph showing the effect on mice of (1) 2.5 mg/kg; (2) 5 mg/kg; (3) 10 mg/kg; 25 mg/kg; and 50 mg/kg of MPH compared with saline on ambulation counts per 10 minutes over an eight-hour session.

FIG. 4 is a graph showing the effect on mice of (1) 1 mg/kg; (2) 3 mg/kg; (3) 10 mg/kg; and 30 mg/kg of Sample D (from Tables 1 and 3) compared with saline on ambulation counts per 10 minutes over an eight-hour session.

### DETAILED DESCRIPTION OF THE INVENTION

Before describing the present invention in detail, it is to be understood that this invention is not limited to particular drug delivery systems, as such may vary. It is also to be understood that the terminology used herein is for the purpose of describing particular embodiments only, and is not intended to be limiting. In describing and claiming the present invention, the following terminology will be used in accordance with the definitions set forth below.

Definitions and Nomenclature:

As used in this specification and the appended claims, the singular forms “a,” “an,” and “the” include plural referents unless the context clearly dictates otherwise.

The term “isomer,” “optical isomer” (an optically active isomer), and “stereoisomer” (a three-dimensional isomer) are used interchangeably and refer to one of two or more molecules having the same number and kind of atoms and hence the same molecular weight, but differing in respect to the arrangement or configuration of the atoms. Where isomers are mirror images of each other, they are called “enantiomers.” Thus, within the context of the MPH analogs of the present invention, an analog with an R,R configuration is an enantiomer to an analog with an S,S configurations; likewise, an analog with an R,S configuration is an enantiomer to an analog with an S,R configuration.

The term “racemate” refers to a composite of equimolar quantities of two enantiomeric species. Within the context of the present invention, compounds with the R,R/S,S configuration are racemates as are compounds with the R,S/S,R configuration.

The terms “active agent,” “drug,” and “pharmacologically active agent” are used interchangeably herein to refer to a chemical material or compound which, when administered to an organism (human or animal) induces a desired pharmacologic effect. Included are derivatives that include pharmacologically acceptable and pharmacologically active salts, esters and amides, as well as prodrugs, conjugates and active metabolites. Analogs of those compounds or classes of compounds specifically mentioned that also induce the desired pharmacologic effect, are also included.

As used herein, the phrase “having the formula” or “having the structure” is not intended to be limiting and is used in the same way that the term “comprising” is commonly used.

The term “alkyl” as used herein refers to a branched or unbranched saturated hydrocarbon group typically although not necessarily containing 1 to about 18 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred lower alkyl substituents contain 3 to 5 carbon atoms, and particularly preferred such substituents contain 4 carbon atoms (e.g., isobutyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkoxy” as used herein intends an alkyl group bound through a single, terminal ether linkage; that is, an “alkoxy” group may be represented as —O--alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred lower alkoxy substituents contain 1 to 3 carbon atoms, and particularly preferred such substituents contain 1 or 2 carbon atoms (i.e., methoxy and ethoxy).

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 6 to 24 carbon atoms, and particularly preferred aryl groups contain 6 to 16, optimally 6 to 12, carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “alkaryl” refers to an aryl group with an alkyl substituent, and the term “aralkyl” refers to an alkyl group with an aryl substituent, wherein “aryl” and “alkyl” are as defined above. Preferred aralkyl groups contain 6 to 24 carbon atoms, and particularly preferred aralkyl groups contain 6 to 16, optimally 6 to 12, carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, 2,4-dimethylphenyl, 2,7-dimethylnaphthyl, 7-cyclooctylnaphthyl, 3-ethyl-cyclopenta-1,4-diene, and the like. The terms “alkaryloxy” and “aralkyloxy” refer to substituents of the formula —OR wherein R is alkaryl or aralkyl, respectively, as just defined.

The term “alicyclic” refers to compounds that are both aliphatic and cyclic, but not aromatic.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur, preferably nitrogen or oxygen. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 24 carbon atoms, preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halogens, hydroxyl, sulfhydryl, C₁-C₂₄ alkoxy, C₅-C₂₄ aryloxy, acyl (including C₂-C₂₄ alkylcarbonyl (—CO-alkyl) and C₆-C₂₄ arylcarbonyl (—CO-aryl)), acyloxy (—O-acyl), C₂-C₂₄ alkoxycarbonyl (—(CO)—O-alkyl), C₆-C₂₄ aryloxycarbonyl (—(CO)—O-aryl), halocarbonyl (—CO)—X where X is halo), carboxy (—COOH), carbamoyl (—(CO)—NH₂), mono-(C₂-C₂₄ alkyl)-substituted carbamoyl (—(CO)—NH(C₁-C₂₄ alkyl)), di-(C₁-C₂₄ alkyl)substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N—(C₁-C₂₄ alkyl), N—(C₆-C₂₄ aryl)-substituted carbamoyl, cyano(—C≡N), formyl (—(CO)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), phosphono (—P(O)(OH)₂), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₅-C₂₄ aryl (preferably C₅-C₁₄ aryl), C₆-C₂₄ alkaryl (preferably C₆-C₁₈ alkaryl), and C₆-C₂₄ aralkyl (preferably C₆-C₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

When referring to a compound of the invention as an active agent, applicants intend the term “compound” or “active agent” to encompass not only the specified molecular entity but also its pharmaceutically acceptable, pharmacologically active analogs, including, but not limited to, salts, esters, amides, prodrugs, conjugates, active metabolites, and other such derivatives, analogs, and related compounds.

By “pharmaceutically acceptable,” as in the recitation of a “pharmaceutically acceptable carrier,” or “pharmaceutically acceptable salt” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”), as in a “pharmacologically active” derivative of an active agent, refers to a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term “pharmaceutically acceptable” is used to refer to an excipient, it implies that the excipient has met the required standards of toxicological and manufacturing testing or that it is on the Inactive Ingredient Guide prepared by the FDA.

The term “patient” as in treatment of “a patient” refers to a human individual suffering from drug addiction, attention deficit disorder, attention deficit hyperactivity disorder, and/or depression.

The terms “treating” and “treatment” as used herein with respect to treatment of a patient refer to a reduction or elimination in the patient's desire and/or craving for the drugs causing addiction, as well as to treatment of a patient for attention deficit disorder, attention deficit hyperactivity disorder, and/or depression.

The terms “effective amount” or “therapeutically effective amount” of an active agent as provided herein to mean an amount of an active agent that is nontoxic, but sufficient to provide the desired therapeutic effect. The exact amount required will vary from subject to subject, depending on the age, weight, and general condition of the subject, the severity of the condition being treated, the judgment of the clinician, and the like. Thus, it is not always possible to specify an exact “effective amount”; however, an appropriate “effective” amount in any individual case may be determined by one of ordinary skill in the art using routine experimentation.

The term “dosage form” denotes any form of a pharmaceutical composition that contains an amount of active agent sufficient to achieve a therapeutic effect with a single administration. The frequency of administration that will provide the most effective results in an efficient manner without overdosing will vary with the characteristics of the particular active agent, including both its pharmacological characteristics and its physical characteristics, such as hydrophilicity.

The MPH Analogs:

In one embodiment of the present invention, the MPH analogs of the present invention are comprised of a compound having the structure of formula (I)

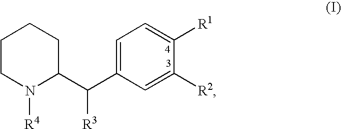

wherein R¹ and R² are independently selected from hydrogen, halogen, alkyl, alkoxy, substituted alkyl, aryl, and aralkyl, with the proviso that at least one of R¹ and R² is other than hydrogen; R³ is selected from alkyl, substituted alkyl, heteroalkyl, substituted heteroalkyl, aryl, aralkyl, substituted aralkyl, heteroaralkyl, and substituted heteroaralkyl; and R⁴ is hydrogen, alkyl, or aralkyl.

In a preferred embodiment of the compound of formula (I), R¹ and R² are independently selected from hydrogen, halogen, C₁-C₆ alkyl, and C₁-C₆ alkoxy; R³ is selected from C₁-C₁₂ alkyl, substituted C₁-C₁₂ alkyl, C₁-C₁₂ heteroalkyl, substituted C₁-C₁₂ heteroalkyl, C₆-C₁₂ aryl, C₆-C₁₆ aralkyl, substituted C₆-C₁₆ aralkyl, C₆-C₁₆ heteroaralkyl, and substituted C₆-C₁₆ heteroaralkyl; and R⁴ is hydrogen, C₁-C₆ alkyl, or C₆-C₁₂ aralkyl.

In a more preferred embodiment of formula (I), R¹ and R² are independently selected from hydrogen and halogen; R³ is selected from C₁-C₆ alkyl, substituted C₁-C₆ alkyl, C₆-C₁₂ aralkyl, and substituted C₆-C₁₂ aralkyl; and R⁴ is hydrogen or CH₃.

In one most preferred embodiment of formula (I), R¹ is hydrogen; R² is chlorine; R³ is C₁-C₆ alkyl or C₆-C₁₂ aralkyl; and R⁴ is hydrogen. In one exemplary compound, R³ is C₁-C₆ alkyl, more preferably isobutyl, and in another exemplary compound R³ is C₆-C₁₂ alicyclic, more preferably cyclopentylmethyl.

In another most preferred embodiment of formula (I), R¹ and R² are chlorine; R³ is C₁-C₆ alkyl; and R⁴ is hydrogen or CH₃. In one exemplary compound, R³ is isobutyl.

The MPH analogs of the present invention may be a mixture of any of four stereoisomers: R,R; S,S; R,S; and S,R. The various stereoisomers of the methylphenidate analogs of the present invention are shown in formulas (Ia) to (Id) as follows: the R,R isomer is shown in formula (Ia); the S,S isomer is shown in formula (Ib); the R,S isomer as shown in formula (Ic); and the S,R isomer is shown in formula (Id).

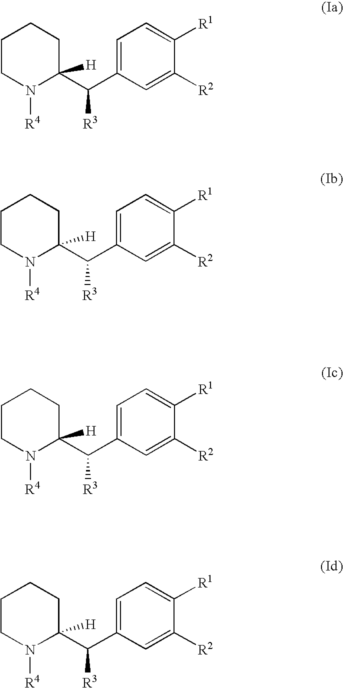

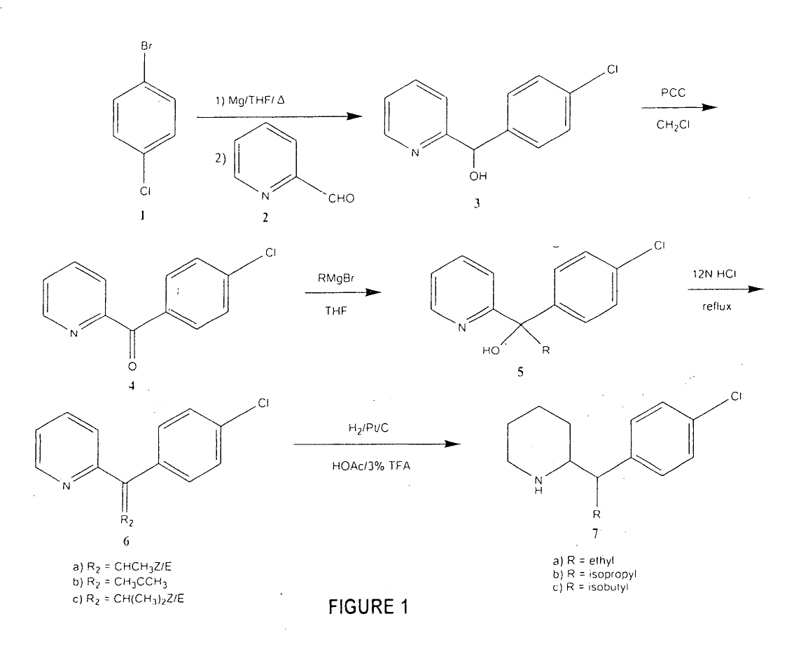

In another embodiment of the present invention, the MPH analogs may be synthesized by the following procedure: converting a substituted bromobenzene compound to a Grignard reagent, followed by condensation with pyridine-2-carboxaldehyde to a pyridin-2yl-methanol; oxidizing the pyridin-2yl-methanol to convert the hydroxyl group to a carbonyl group, thereby providing a pyridin-2yl-methanone; reacting the pyridin-2-yl-methanone with a Grignard reagent RMgBr to replace the carbonyl with a hydroxyl group and an R substituent (identified as R group R³ in Tables 1 and 2) and removing the hydroxyl group by dehydration followed by hydrogenation of the olefin and dearomatization the pyridine ring to a piperidine ring. In a preferred embodiment, the MPH analogs of the invention are synthesized as follows (a) converting 1-chloro-4-bromobenzene into a Grignard reagent with magnesium and tetrahydrofuran; (b) reacting the Grignard reagent with pyridine-2-carboxaldehyde to produce an alcohol; (c) oxidizing the alcohol with pyridinium chlorochromate in methylene chloride to produce a ketone; (d) reacting the ketone with a Grignard reagent to produce an alcohol; (e) dehydrating and refluxing the alcohol with hydrogen chloride to produce an olefin; and (f) hydrogenating the olefin and pyridine to produce an MPH analog according to formula (I), wherein the Grignard reagent of step (d) contains functional R groups for inclusion in the MPH analog provided step (f). In a preferred embodiment, the compound provided in step (f) has the structure of formula (I) as described above. FIG. 1 and Example 1 illustrate and describe the synthesis of three MPH alkyl analogs of the present invention.

Pharmaceutical Formulations and Dosage Forms:

For administration to an individual suffering from drug addiction, attention deficit disorder, attention deficit hyperactivity disorder, or depression, the MPH analogs of the present invention are prepared as pharmaceutical formulations containing a therapeutically effective amount of one or more compounds of formulas (I), (Ia), (Ib), (Ic), (Id), or a pharmaceutically acceptable salt thereof. A pharmaceutically acceptable carrier may also be included, as may other therapeutic ingredients.

Pharmaceutical formulations containing a therapeutically effective amount of the MPH analogs of the present invention may be conveniently presented in unit dosage form and prepared by any of the methods well known in the art of pharmacy. Preferred unit pharmaceutical formulations are those containing an effective dose, or an appropriate fraction thereof, of the active ingredient, or a pharmaceutically acceptable salt thereof. The magnitude of a prophylactic or therapeutic dose typically varies with the nature and severity of the condition to be treated and the route of administration. The dose, and perhaps the dose frequency, will also vary according to the age, body weight, and response of the individual patient. In general, the total daily dose ranges from about 0.1 mg/kg per day to about 30 mg/kg per day, preferably about 1 mg/kg per day to about 20 mg/kg per day, and more preferably, about 3 mg/kg per day to about 10 mg/kg per day, in once or twice daily doses. It is further recommended that children, patients over 65 years old, and those with impaired renal or hepatic function, initially receive low doses and that the dosage is later titrated based on individual responses and blood levels. It may be necessary to use dosages outside these ranges in some cases, as will be apparent to those in the art. Further, it is noted that the clinician or treating physician knows how and when to interrupt, adjust or terminate therapy in conjunction with individual patient's response.

Any suitable route of administration may be employed for providing the patient with an effective dosage of the MPH analogs described herein. Administration can be, for example, oral, parenteral, transdermal, transmucosal (including rectal and vaginal), sublingual, by inhalation, or via an implanted reservoir in a dosage form. The term “parenteral” as used herein is intended to include subcutaneous, intravenous, and intramuscular injection.

Depending on the intended mode of administration, the pharmaceutical formulation may be a solid, semi-solid or liquid, such as, for example, a tablet, a capsule, a caplet, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in REMINGTON: THE SCIENCE AND PRACTICE OF PHARMACY (Easton, Pa.: Mack Publishing Co., 1995). For those compounds that are orally active, oral dosage forms are generally preferred, and include tablets, capsules, caplets, solutions, suspensions and syrups, and may also comprise a plurality of granules, beads, powders, or pellets that may or may not be encapsulated. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like.

Oral dosage forms, whether tablets, capsules, caplets, or particulates, may, if desired, be formulated so as to provide for gradual, sustained release of the active agent over an extended time period. Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material.

Preparations according to this invention for parenteral administration include sterile aqueous and nonaqueous solutions, suspensions, and emulsions. Injectable aqueous solutions contain the active agent in water-soluble form. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The compounds of the invention may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above or it may be a liquid or hydrogel reservoir, or may take some other form. Transdermal drug delivery systems may in addition contain a skin permeation enhancer.

As will be appreciated by those skilled in the art and as described in the pertinent texts and literature, a number of methods are available for preparing drug-containing tablets or other dosage units, which provide a variety of drug release profiles. Such methods include coating a drug or drug-containing composition, increasing the drug's particle size, placing the drug within a matrix, and forming complexes of the drug with a suitable complexing agent.

For example, the pharmaceutical formulations containing the MPH analogs of the present invention may be prepared as delayed release dosage units by coating a drug or a drug-containing composition with a selected membrane coating material, typically although not necessarily a polymeric material. When a coating is used to provide delayed release dosage units, particularly preferred coating materials comprise bioerodible, gradually hydrolyzable and/or gradually water-soluble polymers. The “coating weight,” or relative amount of coating material per dosage unit, generally dictates the time interval between ingestion and drug release.

The active agents in the present compositions and dosage forms may be in the form of a pharmaceutically acceptable salt, ester, amide, prodrug, or other derivative or analog, including active agents modified by appending one or more appropriate functionalities to enhance selected biological properties. Such modifications are known in the art and/or are described in the pertinent texts and literature.

Utility:

The MPH analogs of the present invention have utility in the treatment of drug addiction, attention deficit disorder, attention deficit hyperactivity disorder, and depression. As shown in Example 2 (Tables 3 and 4), the MPH analogs of the present invention act as effective dopamine reuptake blockers. Table 3 and 4 indicate that samples N, E, G, D (in that order) from Table 3 and samples NN and OO from Table 4 are the most potent MPH analogs in that they are particularly effective at binding to the dopamine transporter and blocking dopamine reuptake. Further, as indicated in Table 3, samples B, D, E, F, J, and L also show enhanced selectivity for the dopamine transporter over the norepinephrine transporter (see, values for NE/DEreuptake in Table 3). Since most abused drugs have an effect on the dopamine system, the MPH analogs of the present invention have utility in the treatment of individuals abusing drugs. While the MPH analogs of the present invention have appreciable utility in the treatment of addiction to dopamine reuptake blockers such as cocaine and methylphenidate, the MPH analogs also have utility in the treatment of drug addiction to stimulants, such as amphetamines, as well as drugs that have a secondary effect on the dopamine system, such as opiates, alcohol, and nicotine.

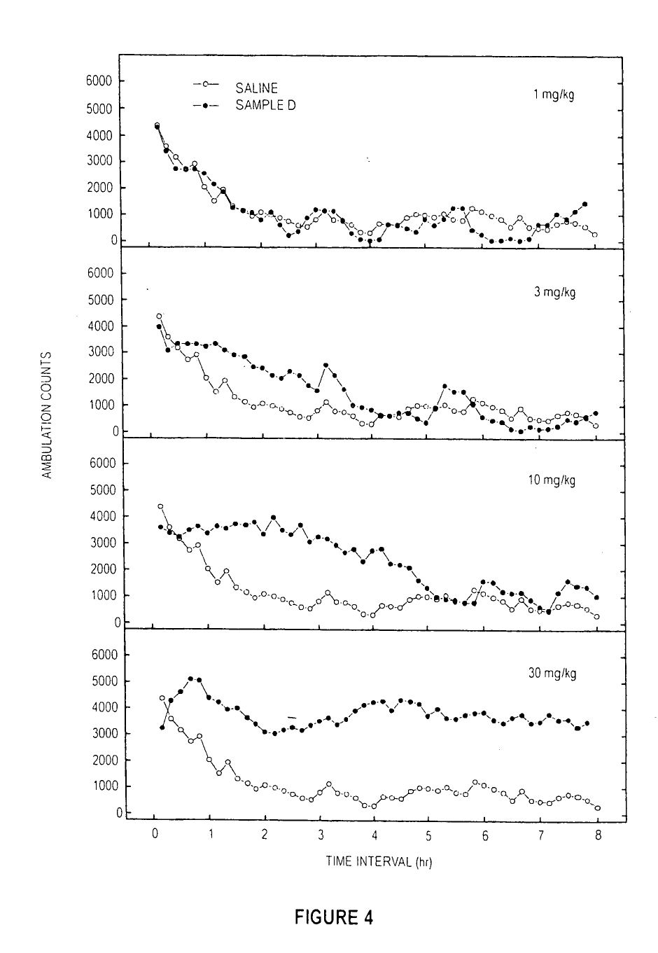

Because of the extended duration of activity of the MPH analogs is well in excess of eight hours (Example 3 and FIG. 4), the addicted individual need only administer the MPH analogs once daily in order to quell the cravings associated with dopamine depletion that occurs with abstinent cocaine addicts. This type of administration is critically important for the treatment of drug addiction, as most addicts do not have a lifestyle that can maintain multiple regimented doses of treatment on a daily basis. When appropriate, it may be preferable to administer lower doses of the MPH analogs to twice-daily dosages.

Because of its long-acting duration and ability to bind to the dopamine transporter and initiate dopamine reuptake, the MPH analogs of the present invention, and in particular samples B, D, E, F, G, J, L, and N from Table 3 and samples NN and OO from Table 4, may be particularly effective as pharmacological agents for the treatment of addiction to dopamine reuptake blockers, such as cocaine, by regulating the amount of dopamine in the afflicted individual's brain.

It is to be understood that while the invention has been described in conjunction with the preferred specific embodiments thereof, that the foregoing description as well as the examples that follow are intended to illustrate and not limit the scope of the invention. Other aspects, advantages, and modifications within the scope of the invention will be apparent to those skilled in the art to which the invention pertains.

All patents and publications mentioned herein, both supra and infra are incorporated by reference in their entireties.

### EXPERIMENTAL

The following examples are put forth so as to provide those of ordinary skill in the art with a complete disclosure and description of how to make and use the compositions of the invention. Efforts have been made to ensure accuracy with respect to numbers (e.g., amounts, temperature, etc.) but some experimental error and deviations should be taken into account. Unless indicated otherwise, parts are parts by weight, temperature is degrees centigrade, and pressure is at or near atmospheric. Unless otherwise indicated, all materials used in the following examples are commercially available products.

### Example 1

### Synthesis of MPH Analogs

The compounds of the present invention were synthesized according to the procedure, which is illustrated schematically in FIG. 1 for three MPH alkyl analogs. Referring to FIG. 1, para-bromochlorobenzene 1 was converted into a Grignard reagent with Mg/THF which was then reacted with the pyridine-2-carboxaldehyde 2 to produce the alcohol 3. The alcohol 3 was oxidized with pyridinium chlorochromate in CH₂Cl₂ to produce the ketone 4. The ketone 4 was then reacted with a Grignard reagent that contains the required R group to produce the alcohol 5. After dehydration with refluxing HCl, the resulting Z and E olefin mixture 6 was hydrogenated with 10% Pt/C in HOAc containing 3% CF₃COOH to produce the final compounds 7 with a ratio of about 40:60 of the R,R/S,S and R,S/S,R racemates for the ethyl compound. The racemates were separated by column chromatography and their relative configurations were determined by x-ray crystallography.

Using the procedure described above and illustrated schematically in FIG. 1, several different MPH analogs were prepared. Table 1 lists R,R/S,S racemates prepared from Formula (I′), which is identical to Formula (I′), but additionally indicates positions 3 and 4 on the phenyl ring of the compound, and Table 2 lists R,S/S,R racemates prepared from Formula (I′).

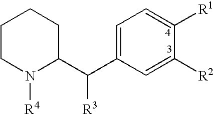

TABLE 1
RR/SS RACEMATES

| SAMPLE NO. | R1 & R2 | R3 | R4 |
|---|---|---|---|
| A | 4-Cl; 3-H | COOCH₃ | H |
| B | 4-Cl; 3-H | ethyl | H |
| C | 4-Cl; 3-H | isopropyl | H |
| D | 4-Cl; 3-H | isobutyl | H |
| E | 4-Cl; 3-H | propyl | H |
| F | 4-Cl; 3-H | neopentyl | H |
| G | 4-Cl; 3-H | butyl | H |
| H | 4-Cl; 3-H | cyclohexylmethyl | H |
| I | 4-Cl; 3-H | phenethyl | H |
| J | 4-Cl; 3-H | benzyl | H |
| K | 4-Cl; 3-H | cyclopentyl | H |
| L | 4-Cl; 3-H | cyclopentylmethyl | H |
| M | 3,4-diCl | COOCH₃ | H |
| N | 3,4-diCl | isobutyl | H |
| O | 3,4-diCl | isobutyl | CH₃ |
| P | 4-isopropyl; 3-H | isobutyl | H |
| Q | 4-Cl; 3-H | 3-pentyl | H |
| R | 4-Cl; 3-H | isopentyl | H |
| S | 4-Cl; 3-H | pentyl | H |
| T | 4-Cl; 3-H | 2-phenylpropyl | H |
| U | 4-Cl; 3-H | 3-phenylpropyl | H |

TABLE 2
RS/SR RACEMATES

| SAMPLE NO. | R1 & R2 | R3 | R4 |
|---|---|---|---|
| AA | 4-Cl; 3-H | COOCH₃ | H |
| BB | 4-Cl; 3-H | ethyl | H |
| CC | 4-Cl; 3-H | isopropyl | H |
| DD | 4-Cl; 3-H | isobutyl | H |
| EE | 4-Cl; 3-H | propyl | H |
| FF | 4-Cl; 3-H | neopentyl | H |
| GG | 4-Cl; 3-H | butyl | H |
| HH | 4-Cl; 3-H | cyclohexylmethyl | H |
| II | 4-Cl; 3-H | phenethyl | H |
| JJ | 4-Cl; 3-H | benzyl | H |
| KK | 4-Cl; 3-H | cyclopentyl | H |
| LL | 4-Cl; 3-H | cyclopentylmethyl | H |
| MM | 3,4-diCl | COOCH₃ | H |
| NN | 3,4-diCl | isobutyl | H |
| OO | 3,4-diCl | isobutyl | CH₃ |
| PP | 4-isopropyl; 3-H | isobutyl | H |
| QQ | 4-Cl; 3-H | 3-pentyl | H |
| RR | 4-Cl; 3-H | isopentyl | H |
| SS | 4-Cl; 3-H | pentyl | H |
| TT | 4-Cl; 3-H | 2-phenylpropyl | H |
| UU | 4-Cl; 3-H | 3-phenylpropyl | H |

### Example 2

### Effectiveness of MPH Analogs on Dopamine Binding and Reuptake

The methylphenidate analogs synthesized according to the procedure set forth in Example 1 were tested in binding and reuptake assays utilizing recombinant human dopamine, norepinephrine, and serotonin transporters stably expressed in human embryonic kidney 293 cells. The binding studies measured the displacement of [¹²⁵I]RTI-55 by the test compounds while the reuptake studies measured the potency of the test compounds in inhibiting the reuptake of the tritiated monoamine neurotransmitters. The potency of the novel compounds in binding to the cloned human monoamine transporters and their potency at inhibiting the binding of dopamine, serotonin, and norepinephrine at their respective transporters are shown in Table 3 for the RR,SS racemates (from Table 1) and in Table 4 for the RS,SR racemates (from Table 2). This table shows the results of the binding affinity (nM) and reuptake inhibition potency (nM) of cocaine, MPH, Samples A-P of Table 1, and Samples AA-PP of Table 2 with human dopamine (DA), serotonin (5-HT), and norepinephrine (NE) transporters as expressed in the human embryonic kidney 293 cells. The assays used for the experiments are described in Eshleman et al., J. PHARMACOL. EXP. THER. 289:877-885 (1999); the assays described in this reference are incorporated herein by reference. Because the R,R/S,S racemates are the active isomers of the MPH analogs of the present invention (comparable to the active threo isomers of MPH), Table 3 includes the ratio of NE/DA reuptake in order to provide a value to indicate the selectivity of the R,R/S,S racemates for the dopamine transporter over the norepinephrine transporter.

TABLE 3
BINDING (Kᵢ, nM) AND REUPTAKE (IC₅₀, nM) STUDIES OF RR/SS RACEMATES

| | DOPAMINE | | SEROTONIN | | NOREPINEPHRINE | | |
|---|---|---|---|---|---|---|---|
| | [I¹²⁵]-RTI- | DA | [I¹²⁵]-RTI- | 5HT | [I¹²⁵]-RTI- | NE | NE/DA |
| SAMPLE NO. | 55 BINDING | REUPTAKE | 55 BINDING | REUPTAKE | 55 BINDING | REUPTAKE | REUPTAKE |
| cocaine | 459 ± 69 | 244 ± 28 | 522 ± 42 | 314 ± 44 | 1970 ± 130 | 238 ± 33 | 0.98 |
| MPH | 180 ± 40 | 190 ± 50 | 40000 ± 8000 | 55000 ± 16000 | 1800 ± 800 | 38 ± 4 | 0.2 |
| (threo- | | | | | | | |
| R, R/S, S) | | | | | | | |
| A | 25 ± 8 | 11 ± 2 | 6000 ± 100 | >9800 | 110 ± 40 | 11 ± 3 | 1.0 |
| B | 37 ± 10 | 23 ± 5 | 7800 ± 800 | 2400 ± 400 | 360 ± 60 | 210 ± 30 | 9.1 |
| C | 46 ± 16 | 32 ± 6 | 5300 ± 1300 | 3300 ± 400 | 810 ± 170 | 51 ± 20 | 1.6 |
| D | 16 ± 4 | 8.6 ± 2.9 | 5900 ± 900 | 490 ± 80 | 840 ± 130 | 120 ± 40 | 14 |
| E | 11 ± 3 | 7.4 ± 0.4 | 2700 ± 600 | 2900 ± 1100 | 200 ± 80 | 50 ± 15 | 6.8 |
| F | 120 ± 40 | 60 ± 2 | 3900 ± 500 | >8300 | 1400 ± 400 | 520 ± 110 | 8.7 |
| G | 7.8 ± 1.1 | 8.2 ± 2.1 | 4300 ± 400 | 4000 ± 400 | 230 ± 30 | 26 ± 7 | 3.2 |
| H | 130 ± 40 | 230 ± 70 | 900 ± 400 | 1000 ± 200 | 4200 ± 200 | 940 ± 140 | 4.1 |
| I | 24 ± 9 | 160 ± 20 | 640 ± 60 | 650 ± 219 | 1800 ± 600 | 680 ± 240 | 4.3 |
| J | 440 ± 110 | 370 ± 90 | 1100 ± 200 | 1100 ± 200 | 2900 ± 800 | 2900 ± 600 | 7.8 |
| K | 36 ± 10 | 27 ± 8.3 | 5700 ± 1100 | 4600 ± 800 | 380 ± 120 | 44 ± 18 | 1.6 |
| L | 9.4 ± 1.5 | 21 ± 1 | 2900 ± 80 | 2100 ± 900 | 1700 ± 600 | 310 ± 40 | 15 |
| M | 1.4 ± 0.1 | 23 ± 3 | 1600 ± 150 | 540 ± 110 | 14 ± 6 | 10 ± 1 | 0.43 |
| N | 1.0 ± 0.5 | 5.5 ± 1.3 | 1600 ± 100 | 1100 ± 300 | 25 ± 9 | 9.0 ± 1 | 1.6 |
| O | 6.6 ± 0.9 | 13 ± 4 | 1300 ± 200 | 1400 ± 500 | 190 ± 60 | 29 ± 3 | 2.2 |
| P | 3300 ± 600 | 4000 ± 400 | 3300 ± 600 | 4700 ± 700 | 2500 ± 600 | 7100 ± 1800 | 1.8 |

TABLE 4
BINDING (Kᵢ, nM) AND REUPTAKE (IC₅₀, nM) STUDIES OF RS/SR RACEMATES

| | DOPAMINE | | SEROTONIN | | NOREPINEPHRINE | |
|---|---|---|---|---|---|---|
| | [I¹²⁵]-RTI- | DA | [I¹²⁵]-RTI- | 5HT | [I¹²⁵]-RTI- | NE |
| SAMPLE NO. | 55 BINDING | REUPTAKE | 55 BINDING | REUPTAKE | 55 BINDING | REUPTAKE |
| cocaine | 459 ± 69 | 244 ± 28 | 522 ± 42 | 314 ± 44 | 1970 ± 130 | 238 ± 33 |
| MPH | 180 ± 40 | 190 ± 50 | 40000 ± 8000 | 55000 ± 16000 | 1800 ± 800 | 38 ± 4 |
| (threo- | | | | | | |
| R, R/S, S) | | | | | | |
| AA | 2000 ± 600 | 2700 ± 1000 | 5900 ± 200 | >10 μM | >6100 | 1400 ± 400 |
| BB | 3500 ± 1000 | 2200 ± 300 | 5700 ± 800 | 2000 ± 600 | >10 μM | |
| CC | 900 ± 320 | 990 ± 280 | >10 μM | | >10 μM | |
| DD | 170 ± 50 | 380 ± 130 | 4300 ± 500 | 540 ± 150 | 4500 ± 1500 | 750 ± 170 |
| EE | 380 ± 40 | 450 ± 60 | 3200 ± 1100 | 1300 ± 7 | 1400 ± 400 | 200 ± 50 |
| FF | 600 ± 40 | 670 ± 260 | 3500 ± 1000 | 1800 ± 600 | >5500 | 730 ± 250 |
| GG | 290 ± 70 | 170 ± 40 | 4800 ± 700 | 3300 ± 600 | 1600 ± 300 | 180 ± 160 |
| HH | 260 ± 30 | 410 ± 60 | 3700 ± 500 | 6400 ± 1300 | 4300 ± 200 | 1700 ± 600 |
| II | 700 ± 90 | 420 ± 140 | 1840 ± 70 | 2100 ± 900 | 2400 ± 700 | 610 ± 150 |
| JJ | 550 ± 60 | 390 ± 60 | 4300 ± 800 | 4700 ± 500 | 4000 ± 800 | >800 |
| KK | 690 ± 140 | 240 ± 30 | 4600 ± 700 | 4200 ± 900 | 3300 ± 800 | 1000 ± 300 |
| LL | 310 ± 80 | 180 ± 20 | 3200 ± 700 | 5600 ± 1400 | 2600 ± 800 | 730 ± 230 |
| MM | 90 ± 14 | 800 ± 110 | 2500 ± 420 | 1100 ± 90 | 4200 ± 1900 | 190 ± 50 |
| NN | 31 ± 11 | 13 ± 3 | 450 ± 40 | 290 ± 60 | 120 ± 30 | 19 ± 3 |
| OO | 44 ± 12 | 45 ± 4 | 1500 ± 300 | 2400 ± 700 | 660 ± 130 | 100 ± 19 |
| PP | >6500 | >9100 | 1700 ± 500 | 1700 ± 100 | 3200 ± 600 | >8700 |

The results of Tables 3 and 4 show that several of the R,R/S,S racemates and Samples NN and OO of the R,S/S,R racemates were considerably more potent at binding to the dopamine transporter and blocking the reuptake of dopamine than was MPH (compare all samples with dopamine binding and reuptake values that are less than that of MPH). Also significant with respect to selectivity of the MPH analogs of the present invention, is the enhanced selectivity of particular samples for the dopamine transporter over the norepinephrine transporter, which is evidenced by comparing the values provided above for dopamine and norepinephrine binding and reuptake. While MPH does not demonstrate enhanced selectivity for the dopamine transporter over the norepinephrine transporter, the MPH analogs of the present invention that do demonstrate enhanced selectivity for the dopamine transporter over the norepinephrine transporter.

Of the R,R/S,S racemates, Samples D and L demonstrated the most selective activity as indicated by their NE/DA reuptake values of 14 and 15, respectively; thus, these two samples were 14-fold and 15-fold more selective in blocking reuptake of dopamine than they were in blocking reuptake of norepinephrine. In addition to Samples D and L, Samples B, E, F, and J also demonstrated enhanced selectivity for the dopamine transporter over the norepinephrine transporter and thus, this set of samples would also be expected to be effective compounds for the treatments described herein. Further, because Samples N, E, G, D show enhanced potency for dopamine binding and dopamine reuptake, this set of samples is also useful for the treatments described herein.

The potency of the binding of Samples NN and OO to the dopamine transporters was surprising and unexpected in that the R,S/S,R racemates were not expected to have activity. More interesting is that the activity of Sample NN appears to be dependent upon the addition of a second Cl to the 3-position of the phenyl ring in addition to the Cl at the 4-position of the phenyl ring (compare to the inactivity of Sample DD, which has one Cl at the 4-position and H at the 3-position of the phenyl ring).

As indicated in the Tables 3 and 4, neither MPH nor the MPH analogs demonstrated significant binding to or reuptake at the serotonin transporter.

### Example 3

### Locomotor Studies for Cocaine, MPH, and Sample E

A dose response study of induced locomotor stimulation was conducted according to the following procedure. The study was conducted using 16 Digiscan locomotor activity testing chambers (40.5×40.5×30.5 cm) (Accuscan, Columbus, Ohio) housed in sets of two, within sound-attenuating chambers. A panel of infrared beams (16 beams) and corresponding photodetectors were located in the horizontal direction along the sides of each activity chamber. A 7.5 watt incandescent light above each chamber provided dim illumination. Fans provided an 80-dB ambient noise level within the chamber. Separate groups of eight non-habituated male Swiss-Webster mice (Hsd:ND4, aged 2-3 months) were injected via the intraperitoneal (“IP”) route with either 0.9% saline, deionized water, cocaine, MPH, or Sample D (from Examples 1 ands 2) immediately prior to locomotor activity testing. In all studies, ambulatory activity (interruption of photocell beams) was measured for 8 hours within 10 minute periods, beginning at 0880 hours (two hours after lights on). Testing was conducted with one mouse per activity chamber.

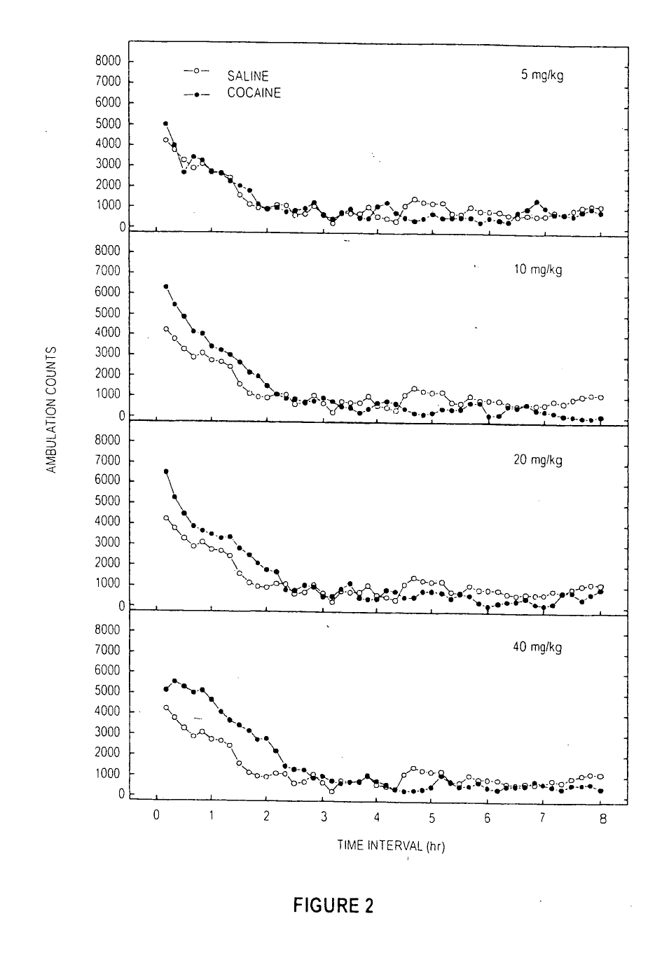

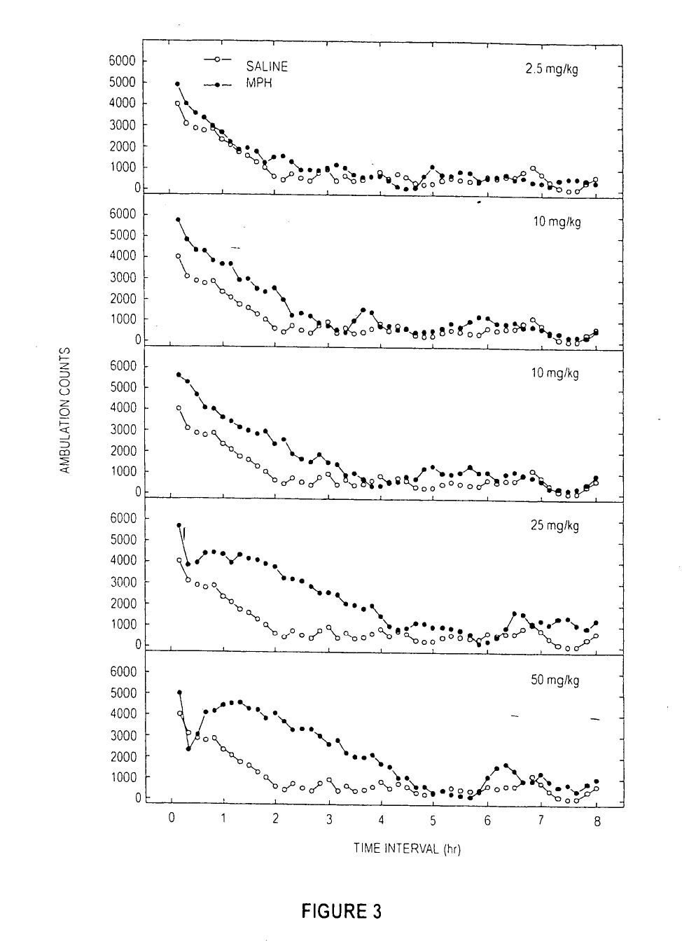

FIGS. 2-4 show average ambulation counts per 10 min as a function of time (0-8 hr) and dose of cocaine versus saline (FIG. 2), MPH versus saline (FIG. 3), and Sample D versus saline (FIG. 4) in doses as indicated in the figures.

As shown in FIG. 2, treatment with cocaine resulted in time-dependent stimulation of locomotor activity in doses from 10 to 40 mg/kg. Stimulant effects of 10, 20, and 40 mg/kg occurred within 10 minutes following injection and lasted up to 2 hours. Maximal stimulant effects were evident during the first 30 minutes following 20 mg/kg cocaine.

As shown in FIG. 3, treatment with MPH resulted in time-dependent stimulation of locomotor activity in doses from 5 to 50 mg/kg. The stimulant effects at 5, 10, 25, and 50 mg/kg occurred within 10 minutes following injection and lasted up to 4 hours. The ambulation count profiles at 5 and 10 mg/kg of MPH were similar as were the ambulation count profiles for 25 and 50 mg/kg MPH; however, the ambulation count profile at the higher doses (i.e., 25 and 50 mg/kg) showed heightened ambulation counts between 1-4 hours while the lower doses (i.e., 5 and 10 mg/kg) showed decreasing ambulation counts during the same period of time.

As shown in FIG. 4, treatment with Sample D resulted in time-dependent stimulation of locomotor activity in doses from 3 to 30 mg/kg. The stimulant effects at 3, 10, and 30 mg/kg did not occur until at least 20 minutes following injection and lasted up to 4 hours at 3 mg/kg, 5 hours at 10 mg/kg and well over 8 hours at 30 mg/kg of Sample D. The lack of enhanced locomotor activity at 20 minutes suggests that the compound has a slow onset. The continued activity of the mice for up to 8 hours upon treatment with Sample D at 30 mg/kg demonstrates that Sample D has a very long duration of action when compared against both cocaine and MPH.

We claim:

1. A compound having the structure of formula (I) 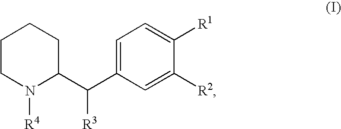
wherein:
R¹ and R² are independently selected from hydrogen, halogen, alkyl, alkoxy, substituted alkyl, aryl, and aralkyl, with the proviso that at least one of R¹ and R² is other than hydrogen;
R³ is selected from C₄-C₁₈ alkyl, substituted alkyl, heteroalkyl, substituted heteroalkyl, aryl, alicyclic, aralkyl, substituted aralkyl, heteroaralkyl, and substituted heteroaralkyl; and
R⁴ is hydrogen, alkyl, or aralkyl.

2. The compound of claim 1 comprised of R,R/S,S racemates of the compound of formula (I).

3. The compound of claim 1 comprised of R,S/S,R racemates of the compound of formula (I).

4. The compound of claim 1, wherein:
R¹ and R² are independently selected from hydrogen, halogen, C₁-C₆ alkyl, and C₁-C₆ alkoxy;
R³ is selected from C₄-C₁₂ alkyl, substituted C₁-C₁₂ alkyl, C₁-C₁₂ heteroalkyl, substituted C₁-C₁₂ heteroalkyl, C₆-C₁₂ aryl, C₆-C₁₂ alicyclic, C₆-C₁₆ aralkyl, substituted C₆-C₁₆ aralkyl, C₆-C₁₆ heteroaralkyl, and substituted C₆-C₁₆ heteroaralkyl; and
R⁴ is hydrogen, C₁-C₆ alkyl, or C₆-C₁₂ aralkyl.

5. The compound of claim 4, wherein:
R¹ and R² are independently selected from hydrogen and halogen;
R³ is selected from C₄-C₈ alkyl, substituted C₁-C₆ alkyl, C₆-C₁₂ alicyclic, C₆-C₁₂ aralkyl, and substituted C₆-C₁₂ aralkyl; and
R⁴ is hydrogen or CH₃.

6. The compound of claim 5, wherein:
R¹ is hydrogen;
R² is chlorine;
R³is s C₄-C₈ alkyl or C₆-C₁₂ alicyclic; and
R⁴ is hydrogen.

7. The compound of claim 6, wherein R³ is C₄-C₈ alkyl.

8. The compound of claim 7, wherein R³ is isobutyl.

9. The compound of claim 6, wherein R³ is C₆-C₁₂ alicyclic.

10. The compound of claim 9, wherein R³ is cyclopentylmethyl.

11. The compound of claims 8 or 10 comprised of R,R/S,S racemates of the compound of formula (I).

12. The compound of claim 4, wherein:
R¹ and R² are chlorine;
R³ is C₁-C₆ alkyl; and
R⁴ is hydrogen or CH₃.

13. The compound of claim 12, wherein R³ is isobutyl.

14. The compound of claim 13 comprised of R,S/S,R racemates of the compound of formula (I).

15. A pharmaceutical composition for treating an individual suffering from drug addiction, attention deficit disorder, attention deficit hyperactivity disorder, or depression, the composition comprising a therapeutically effective amount of the compound of formula (I) and a pharmaceutically acceptable carrier: 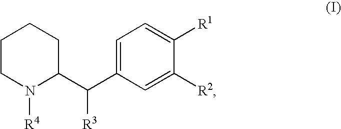
wherein:
R¹ and R² are independently selected from hydrogen, halogen, alkyl, alkoxy, substituted alkyl, aryl, and aralkyl, with the proviso that at least one of R¹ and R² is other than hydrogen;
R³ is selected from C₁-C₁₈ alkyl, substituted alkyl, heteroalkyl, substituted heteroalkyl, aryl, alicyclic, aralkyl, substituted aralkyl, heteroaralkyl, and substituted heteroaralkyl; and
R⁴ is hydrogen, alkyl, or alkaryl.

16. The compound of claim 15 comprised of R,R/S,S racemates of the compound of formula (I).

17. The compound of claim 15 comprised of R,S/S,R racemates of the compound of formula (I).

18. The pharmaceutical composition of claim 15, wherein:
R¹ and R² are independently selected from hydrogen, halogen, C₁-C₆ alkyl, and C₁-C₆ alkoxy;
R³ is selected from C₁-C₁₂ alkyl, substituted C₁-C₁₂ alkyl, C₁-C₁₂ heteroalkyl, substituted C₁-C₁₂ heteroalkyl, C₆-C₁₂ aryl, C₆-C₁₂ alicyclic, C₆-C₁₆ aralkyl, substituted C₆-C₁₆ aralkyl, C₆-C₁₆ heteroaralkyl, and substituted C₆-C₁₆ heteroaralkyl; and
R⁴ is hydrogen, C₁-C₆ alkyl, or C₆-C₁₂ aralkyl.

19. The pharmaceutical composition of claim 18, wherein:
R¹ and R² are independently selected from hydrogen and halogen;
R³ is selected from C₁-C₆ alkyl, substituted C₁-C₆ alkyl, C₆-C₁₂ alicyclic, C₆-C₁₂ aralkyl, and substituted C₆-C₁₂ aralkyl; and
R⁴ is hydrogen or CH₃.

20. The pharmaceutical composition of claim 19, wherein:
R¹ is hydrogen;
R² is chlorine;
R³ is C₁-C₆ alkyl or C₆-C₁₂ alicyclic; and
R⁴ is hydrogen.

21. The pharmaceutical composition of claim 20, wherein R³ is C₁-C₆ alkyl.

22. The pharmaceutical composition of claim 21, wherein R³ is isobutyl.

23. The pharmaceutical composition of claim 20, wherein R³ is C₆-C₁₂ alicyclic.

24. The pharmaceutical composition of claim 23, wherein R³ is cyclopentylmethyl.

25. The pharmaceutical composition of claims 22 or 24 comprised of R,R/S,S racemates of the compound of formula (I).

26. The pharmaceutical composition of claim 19, wherein:
R¹ and R² are chlorine;
R³ is C₁-C₆ alkyl; and
R⁴ is hydrogen or CH₃.

27. The pharmaceutical composition of claim 26, wherein R³ is isobutyl.

28. The pharmaceutical composition of claim 27 comprised of R,S/S,R racemates of the compound of formula (I).

29. The pharmaceutical composition of claim 15, used to treat an individual suffering from addiction to a drug that is a dopamine reuptake blocker.

30. The pharmaceutical composition of claim 29, wherein the drug is cocaine.

31. The pharmaceutical composition of claim 29, wherein the drug is methylphenidate.

32. The pharmaceutical composition of claim 15, wherein the drug is an amphetamine.

33. The pharmaceutical composition of claim 15, wherein the composition is administered orally.

34. The pharmaceutical composition of claim 33, wherein the composition is administered once a day.

35. The pharmaceutical composition of claim 33, wherein the composition is administered twice daily.

36. A method for treating an individual suffering from drug addiction, attention deficit disorder, attention deficit hyperactivity disorder, or depression, comprising administering to the individual a therapeutically effective amount of a compound of formula (I) 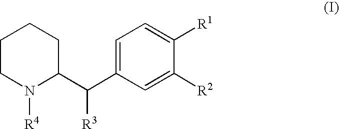
wherein:
R¹ and R² are independently selected from hydrogen, halogen, alkyl, alkoxy, substituted alkyl, aryl, and aralkyl, with the proviso that at least one of R¹ and R² is other than hydrogen;
R³ is selected from C₁-C₁₈ alkyl, substituted alkyl, heteroalkyl, substituted heteroalkyl, aryl, alicyclic, aralkyl, substituted aralkyl, heteroaralkyl, and substituted heteroaralkyl; and
R⁴ is hydrogen, alkyl, or aralkyl.

37. The method of claim 36 comprised of R,R/S,S racemates of the compound of formula (I).

38. The method of claim 36 comprised of R,S/S,R racemates of the compound of formula (I).

39. The method of claim 36, wherein:
R¹ and R² are independently selected from hydrogen, halogen, C₁-C₆ alkyl, and C₁-C₆ alkoxy;
R³ is selected from C₁-C₁₂ alkyl, substituted C₁-C₁₂ alkyl, C₁-C₁₂ heteroalkyl, substituted C₁-C₁₂ heteroalkyl, C₆-C₁₂ aryl, C₆-C1₂ alicyclic, C₆-C₁₆ aralkyl, substituted C₆-C₁₆ aralkyl, C₆-C₁₆ heteroaralkyl, and substituted C₆-C₁₆ heteroaralkyl; and
R⁴ is hydrogen, C₁-C₆ alkyl, or C₆-C₁₂ aralkyl.

40. The method of claim 39, wherein:
R¹ and R² are independently selected from hydrogen and halogen;
R³ is selected from C₁-C₆ alkyl, substituted C₁-C₆ alkyl, C₆-C₁₂ alicyclic, C₆-C₁₂ aralkyl, and substituted C₆-C₁₂ aralkyl; and
R⁴ is hydrogen or CH₃.

41. The method of claim 40, wherein:
R¹ is hydrogen;
R² is chlorine;
R³ is s C₁-C₆ alkyl or C₆-C₁₂ alicyclic; and
R⁴ is hydrogen.

42. The method of claim 41, wherein R³ is C₁-C₆ alkyl.

43. The method of claim 42, wherein R³ is isobutyl.

44. The method of claim 41, wherein R³ is C₆-C₁₂ alicyclic.

45. The method of claim 44, wherein R³ is cyclopentylmethyl.

46. The method of claims 42 or 44 comprised of R,R/S,S racemates of the compound of formula (I).

47. The method of claim 40, wherein:
R¹ and R² are chlorine;
R³ is C₁-C₆ alkyl; and
R⁴ is hydrogen or CH₃.

48. The method of claim 47, wherein R³ is isobutyl.

49. The method of claim 48, comprised of R,S/S,R racemates of the compound of formula (I).

50. The method of claim 36, used to treat an individual suffering from addiction to a dopamine reuptake blocker.

51. The method of claim 50, wherein the dopamine reuptake blocker is cocaine.

52. The method of claim 50, wherein the dopamine reuptake blocker is methylphenidate.

53. The method of claim 36, used to treat an individual suffering from addiction to amphetamines.

54. A method of synthesizing a compound for the treatment of drug addiction, attention deficit disorder, attention deficit hyperactivity disorder, or depression comprising the steps of:
(a) converting 1-chloro-4-bromobenzene into a Grignard reagent with magnesium and tetrahydrofuran;
(b) reacting the Grignard reagent with pyridine-2-carboxaldehyde to produce an alcohol;
(c) oxidizing the alcohol with pyridinium chlorochromate in methylene chloride to produce a ketone;
(d) reacting the ketone with a Grignard reagent to produce an alcohol;
(e) dehydrating and refluxing the alcohol with hydrogen chloride to produce an olefin;
(f) hydrogenating the olefin and pyridine to produce the compound,
wherein the Grignard reagent of step (d) contains functional R groups for inclusion in the compound prepared in step (f).

55. The method of claim 54, wherein the compound provided in step (f) has the structure of formula (I) 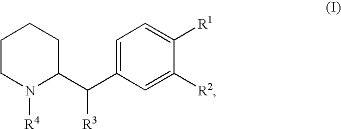
wherein:
R¹ and R² are independently selected from hydrogen, halogen, alkyl, alkoxy, substituted alkyl, aryl, and aralkyl, with the proviso that at least one of R¹ and R² is other than hydrogen;
R³ is selected from alkyl, substituted alkyl, heteroalkyl, substituted heteroalkyl, aryl, alicyclic, aralkyl, substituted aralkyl, heteroaralkyl, and substituted heteroaralkyl; and
R⁴ is hydrogen, alkyl, or aralkyl.

56. The method of claim 54, wherein:
R¹ and R² are independently selected from hydrogen, halogen, C₁-C₆ alkyl, and C₁-C₆ alkoxy;
R³ is selected from C₁-C₁₂ alkyl, substituted C₁-C₁₂ alkyl, C₁-C₁₂ heteroalkyl, substituted C₁-C₁₂ heteroalkyl, C₆-C₁₂ aryl, C₆-C₁₂ alicyclic, C₆-C₁₆ aralkyl, substituted C₆-C₁₆ aralkyl, C₆-C₁₆ heteroaralkyl, and substituted C₆-C₁₆ heteroaralkyl; and
R⁴ is hydrogen, C₁-C₆ alkyl, or C₆-C₁₂ aralkyl.

57. The method of claims 54 or 55, wherein the compound provided in step (f) is comprised of R,R/S,S racemates of the compound of formula (I).

58. The method of claims 54 or 55, wherein the compound provided in step (f) is comprised of R,S/S,R racemates of the compound of formula (I).
